SEQUENCE LISTING (6 sequences)

SEQ ID NO: 1 - DNA, 32 bp, Artificial sequence
  AR Forward Primer
ccatcttgtcgtcttcggaaatgttatgaagc

SEQ ID NO: 2 - DNA, 25 bp, Artificial sequence
  AR Reverse Primer
agcttctgggttgtctcctcagtgg

SEQ ID NO: 3 - DNA, 26 bp, Artificial sequence
  AR-V7 Forward Primer
tgtcactatggagctctcacatgtgg

SEQ ID NO: 4 - DNA, 27 bp, Artificial sequence
  AR-V7 Reverse Primer
ctgtggatcagctactaccttcagctc

SEQ ID NO: 5 - DNA, 20 bp, Artificial sequence
  Beta Actin Forward Primer
tctacaatgagctgcgtgtg

SEQ ID NO: 6 - DNA, 20 bp, Artificial sequence
  Beta Actin Reverse Primer
agcacacagcatgaacttgg

(19) United States
(12) Patent Application Publication
(10) Pub. No.: US 2022/0071978 A1
(43) Pub. Date: Mar. 10, 2022

(54) CANCER TREATMENT PHARMACEUTICAL COMPOSITION CONTAINING CDK INHIBITOR

(71) Applicant: SUMITOMO DAINIPPON PHARMA CO., LTD., Osaka-shi, Osaka (JP)

(72) Inventor: Eijiro Nakamura, Kyoto-shi, Kyoto (JP)

(21) Appl. No.: 17/417,727

(22) PCT Filed: Dec. 26, 2019
(86) PCT No.: PCT/JP2019/051310

§ 371 Date: Jun. 23, 2021

(30) Foreign Application Priority Data
Dec. 27, 2018 (JP) 2018-244174

(51) Int. Cl. A61K 31/453; G01N 33/68; A61P 35/00

(52) U.S. Cl. CPC A61K 31/453; G01N 2800/52; A61P 35/00; G01N 33/6854

(57) ABSTRACT

Provided is a cancer treatment pharmaceutical composition that contains a CDK inhibitor. A pharmaceutical composition that includes a CDK inhibitor and is for treating cancers that demonstrate resistance to androgen removal therapy. The CDK inhibitor includes alvocidib or a pharmaceutically acceptable salt thereof. The cancers are cancers that demonstrate treatment resistance to androgen receptor antagonists and/or androgen synthesis inhibitors. A cancer treatment composition that includes alvocidib or a pharmaceutically acceptable salt thereof as an active ingredient and is to be administered to subjects that have enhanced androgen receptor phosphorylation.

### TECHNICAL FIELD

The present invention relates to a pharmaceutical composition for treating or preventing cancer that exhibits resistance to androgen deprivation therapy (e.g., cancer that exhibits resistance to an androgen receptor antagonist and/or an androgen synthesis inhibitor), comprising a CDK inhibitor as an active ingredient. The present invention also provides a method of selecting a patient on whom a specific CDK inhibitor exhibits efficacy.

### BACKGROUND ART

Specific hormones including steroid hormones are known to be involved in cell proliferation and have a significant effect on oncogenesis or cancer metastasis.

For example, prostate cancer is well known to be exacerbated by male hormones (androgens) including testosterone and androsterone. This is a result of androgen secreted from the testis or adrenal gland acting on an androgen receptor (AR) of a prostate cancer cell, inducing cell proliferation and cancer differentiation/proliferation.

An androgen receptor is a type of nuclear receptor comprised of a “ligand binding domain”, a “DNA binding domain”, and an “N-terminal domain”. When an androgen hormone binds to a ligand binding domain of an androgen receptor within the cytoplasm, the androgen receptor is activated and translocates into the nucleus. The androgen receptor translocated into the nucleus binds to an AR binding domain on a DNA and activates transcription of a specific target gene. It is known that such activation of transcription increases the expression of prostate specific antigens (PSA). Prostate specific antigens are broadly used as a tumor marker for prostate cancer. An increase in the PSA concentration of a prostate cancer patient means that intracellular androgen receptor transcription activity is elevated.

It was reported recently that androgen receptor-mediated androgen signals are deeply involved in not only prostate cancer, but also in a plurality of types of cancer such as bladder cancer (Non Patent Literature 1), ovarian cancer (Non Patent Literature 2), and breast cancer (Non Patent Literature 3).

Examples of methods of treating cancer that exhibits androgen dependency described above include a therapeutic method for suppressing production or secretion of androgen and blocking androgen from binding to an androgen receptor (androgen deprivation therapy). For example, surgical castration, as well as administration of an LH-RH agonist (goserelin, leuprorelin, or the like), flutamide, bicalutamide, nilutamide, or the like are performed against prostate cancer in order to suppress androgen secretion from the testes.

While a lesion contracts momentarily with such androgen deprivation therapy, 90% or more of the cases face a significant problem of acquiring resistance to such a therapy, resulting in recurrence or relapse after about 2 years. For prostate cancer, cancer that exhibits resistance to such androgen deprivation therapy is referred to as “Castration Resistant Prostate Cancer (CRPC)”.

### CITATION LIST

### Patent Literature

[PTL 1] International Publication No. WO 2016/187316
[PTL 2] International Publication No. WO 2018/094275

### Non Patent Literature

[NPL 1] Koji Izumi et al. Oncotarget 5: 12665-12674. (2014)
[NPL 2] Haiyan Zhu et al. Oncotarget 8: 29395-29405. (2017)
[NPL 3] Nicolas Diaz-Chico et al. Journal of Steroid Biochemistry & Molecular Biology 105: 1-15. (2007)

### SUMMARY OF INVENTION

### Solution to Problem

The present invention provides a novel application of a CDK inhibitor. The present invention also provides a method of selecting a patient on whom a specific CDK inhibitor exhibits efficacy. The present invention further provides a pharmaceutical composition comprising a CDK inhibitor as an active ingredient to the selected patient.

As a result of diligent studies, the inventors completed the present invention by finding that alvocidib or a pharmaceutically acceptable salt (hereinafter, also referred to as the “compound of the invention”) exhibits a significant effect of suppressing cancer cell proliferation on “cancer that exhibits resistance to an androgen receptor antagonist or an androgen synthesis inhibitor”.

More specifically, the inventors established “AILNCaP14 cell line” and “AILNCaP15 cell line”, which reflect the pathological condition of castration resistant prostate cancer that exhibits therapeutic resistance to androgen receptor antagonists from “androgen dependent prostate cancer cell line (LNCaP cell line)”. The present invention was completed by studying the effect of alvocidib on these cell lines and finding a significant and different effect of alvocidib inhibiting phosphorylation of a specific residue of an androgen receptor of such cell lines and inhibiting nuclear translocation of the androgen receptor to suppress cell proliferation potently and to a low concentration.

Specifically, the present invention is the following.

### [Item 1]

A pharmaceutical composition for use in treating cancer that exhibits therapeutic resistance to an androgen receptor antagonist and/or an androgen synthesis inhibitor, comprising alvocidib or a pharmaceutically acceptable salt thereof.

### [Item 2]

The pharmaceutical composition of item 1, wherein the cancer is cancer that exhibits resistance to androgen deprivation therapy.

### [Item 3]

The pharmaceutical composition of item 1 or 2, wherein the cancer is at least one type of cancer selected from the group consisting of acute leukemia, chronic lymphocytic leukemia, chronic myelogenous leukemia, polycythemia vera, malignant lymphoma, plasma cell tumor, multiple myeloma, myelodysplastic syndrome, brain tumor, head and neck cancer, esophageal cancer, thyroid cancer, small cell lung cancer, non-small cell lung cancer, thymoma/thymic carcinoma, breast cancer, gastric cancer, gallbladder/bile duct cancer, liver cancer, hepatocellular carcinoma, pancreatic cancer, colon cancer, rectal cancer, anal cancer, gastrointestinal stromal tumor, choriocarcinoma, endometrial cancer, cervical cancer, ovarian cancer, bladder cancer, prostate cancer, urothelial cancer, renal cancer, renal cell cancer, testicular tumor, testicular germ cell tumor, ovarian germ cell tumor, Wilms tumor, skin cancer, malignant melanoma, neuroblastoma, osteosarcoma, Ewing sarcoma, and soft tissue sarcoma.

### [Item 4]

The pharmaceutical composition of any one of items 1 to 3, wherein the cancer is prostate cancer, breast cancer, ovarian cancer, or bladder cancer.

### [Item 5]

The pharmaceutical composition of any one of items 1 to 4, wherein the cancer is prostate cancer.

### [Item 6]

The pharmaceutical composition of any one of items 1 to 5, wherein the cancer is castration resistant prostate cancer.

### [Item 7]

The pharmaceutical composition of any one of items 1 to 6, wherein the cancer is characterized by expressing a mutant androgen receptor.

### [Item 8]

The pharmaceutical composition of item 7, wherein the mutant androgen receptor is a splicing variant of an androgen receptor.

### [Item 9]

The pharmaceutical composition of item 7 or 8, wherein the mutant androgen receptor is a splicing variant AR-V7, AR-V12, or AR-V567es.

### [Item 10]

The pharmaceutical composition of any one of items 7 to 9, wherein the mutant androgen receptor is a splicing variant AR-V7.

### [Item 11]

The pharmaceutical composition of any one of items 1 to 10, wherein the androgen receptor antagonist is enzalutamide.

### [Item 12]

The pharmaceutical composition of any one of items 1 to 11, wherein the androgen synthesis inhibitor is abiraterone.

### [Item 13]

The pharmaceutical composition of any one of items 1 to 12, wherein the active ingredient is alvocidib or a hydrochloric acid salt thereof.

### [Item 14]

The pharmaceutical composition of any one of items 1 to 13, wherein the active ingredient is alvocidib.

### [Item 15]

The pharmaceutical composition of any one of items 1 to 14, wherein the cancer is cancer with elevated phosphorylation of serine 81, and serine 210 or serine 213 of an androgen receptor.

### [Item 16]

A composition for use in treating cancer administered to a patient with a serum testosterone concentration reduced to a castration level by castration and/or drug therapy, comprising alvocidib or a pharmaceutically acceptable salt thereof as an active ingredient.

### [Item 17]

A composition for use in treating cancer administered to a subject with elevated phosphorylation of an androgen receptor, comprising alvocidib or a pharmaceutically acceptable salt thereof as an active ingredient.

### [Item 18]

The composition of item 17, wherein the subject with elevated phosphorylation of an androgen receptor is determined by steps comprising:

(1) quantifying phosphorylation of an androgen receptor of a cancer cell acquired from the subject;
(2) comparing an amount of phosphorylation quantified in (1) with the amount of phosphorylation in a cell collected from a healthy individual (hereinafter, referred to as a control value); and
(3) determining that phosphorylation is elevated when the amount of phosphorylation quantified in (1) is greater than the control value based on a result of (2).

### [Item 19]

The composition of item 18, wherein the amounts of phosphorylation in steps (1) and (2) are measured using an anti-androgen receptor antibody.

### [Item 20]

The composition of item 19, wherein the amounts of phosphorylation in steps (1) and (2) are measured using an anti-androgen receptor antibody as a primary antibody, and further using an anti-beta actin antibody.

### [Item 21]

The composition of any one of items 17 to 20, wherein the phosphorylation of an androgen receptor is phosphorylation of serine 81, and serine 210 or serine 213 of the androgen receptor.

### [Item 22]

The composition of any one of items 17 to 21, wherein the active ingredient is alvocidib or a pharmaceutically acceptable salt thereof.

### [Item 23]

The composition of any one of items 17 to 22, wherein the cancer is at least one type of cancer selected from the group consisting of acute leukemia, chronic lymphocytic leukemia, chronic myelogenous leukemia, polycythemia vera, malignant lymphoma, plasma cell tumor, multiple myeloma, myelodysplastic syndrome, brain tumor, head and neck cancer, esophageal cancer, thyroid cancer, small cell lung cancer, non-small cell lung cancer, thymoma/thymic carcinoma, breast cancer, gastric cancer, gallbladder/bile duct cancer, liver cancer, hepatocellular carcinoma, pancreatic cancer, colon cancer, rectal cancer, anal cancer, gastrointestinal stromal tumor, choriocarcinoma, endometrial cancer, cervical cancer, ovarian cancer, bladder cancer, prostate cancer, urothelial cancer, renal cancer, renal cell cancer, testicular tumor, testicular germ cell tumor, ovarian germ cell tumor, Wilms tumor, skin cancer, malignant melanoma, neuroblastoma, osteosarcoma, Ewing sarcoma, and soft tissue sarcoma.

### [Item 24]

The composition of any one of items 17 to 23, wherein the cancer is prostate cancer, breast cancer, ovarian cancer, or bladder cancer.

### [Item 25]

The composition of any one of items 17 to 24, wherein the cancer is prostate cancer.

### [Item 26]

The composition of any one of items 17 to 25, wherein the prostate cancer is castration resistant prostate cancer.

### [Item 27]

The composition of any one of items 17 to 26, wherein the cancer is characterized by expressing a mutant androgen receptor.

### [Item 28]

The composition of item 27, wherein the mutant androgen receptor is a splicing variant of an androgen receptor.

### [Item 29]

The composition of item 27 or 28, wherein the mutant androgen receptor is a splicing variant AR-V7, AR-V12, or AR-V567es.

### [Item 30]

The composition of any one of items 27 to 29, wherein the mutant androgen receptor is a splicing variant AR-V7.

### [Item 31]

The composition of any one of items 17 to 30, wherein the cancer is cancer that exhibits therapeutic resistance to an androgen receptor antagonist and/or an androgen synthesis inhibitor.

### [Item 32]

The composition of item 31, wherein the androgen receptor antagonist is enzalutamide.

### [Item 33]

The composition of item 31 or 32, wherein the androgen synthesis inhibitor is abiraterone.

### [Item 34]

A method of predicting efficacy of alvocidib or a pharmaceutically acceptable salt thereof, characterized by measuring phosphorylation of an androgen receptor of a subject.

### [Item 35]

The method of item 34, wherein the measurement of phosphorylation of an androgen receptor is determined by steps comprising:

(1) quantifying phosphorylation of a cancer cell acquired from the subject;
(2) comparing an amount of phosphorylation quantified in (1) with the amount of phosphorylation in a cell collected from a healthy individual (hereinafter, referred to as a control value); and
(3) determining whether phosphorylation is elevated when the amount of phosphorylation quantified in (1) is greater than the control value based on a result of (2).

### [Item 36]

The method of item 35, wherein the amounts of phosphorylation in steps (1) and (2) are measured using an anti-androgen receptor antibody.

### [Item 37]

The method of item 36, wherein the amounts of phosphorylation in steps (1) and (2) are measured using an anti-androgen receptor antibody as a primary antibody, and further using an anti-beta actin antibody.

### [Item 38]

The method of any one of items 34 to 37, wherein the phosphorylation of an androgen receptor is phosphorylation of serine 81, and serine 210 or serine 213 of the androgen receptor.

### [Item 39]

The method of any one of items 34 to 38, wherein the cancer is at least one type of cancer selected from the group consisting of acute leukemia, chronic lymphocytic leukemia, chronic myelogenous leukemia, polycythemia vera, malignant lymphoma, plasma cell tumor, multiple myeloma, myelodysplastic syndrome, brain tumor, head and neck cancer, esophageal cancer, thyroid cancer, small cell lung cancer, non-small cell lung cancer, thymoma/thymic carcinoma, breast cancer, gastric cancer, gallbladder/bile duct cancer, liver cancer, hepatocellular carcinoma, pancreatic cancer, colon cancer, rectal cancer, anal cancer, gastrointestinal stromal tumor, choriocarcinoma, endometrial cancer, cervical cancer, ovarian cancer, bladder cancer, prostate cancer, urothelial cancer, renal cancer, renal cell cancer, testicular tumor, testicular germ cell tumor, ovarian germ cell tumor, Wilms tumor, skin cancer, malignant melanoma, neuroblastoma, osteosarcoma, Ewing sarcoma, and soft tissue sarcoma.

### [Item 40]

The method of any one of items 34 to 39, wherein the cancer is prostate cancer, breast cancer, ovarian cancer, or bladder cancer.

### [Item 41]

The method of any one of items 34 to 40, wherein the cancer is prostate cancer.

### [Item 42]

The method of item 41, wherein the prostate cancer is castration resistant prostate cancer.

### [Item 43]

The method of any one of items 34 to 42, wherein the cancer is characterized by expressing a mutant androgen receptor.

### [Item 44]

The method of item 43, wherein the mutant androgen receptor is a splicing variant of an androgen receptor.

### [Item 45]

The method of item 43 or 44, wherein the mutant androgen receptor is a splicing variant AR-V7, AR-V12, or AR-V567es.

### [Item 46]

The method of any one of items 43 to 45, wherein the mutant androgen receptor is a splicing variant AR-V7.

### [Item 47]

The method of any one of items 34 to 46, wherein the cancer is cancer that exhibits therapeutic resistance to an androgen receptor antagonist and/or an androgen synthesis inhibitor.

### [Item 48]

The method of item 47, wherein the androgen receptor antagonist is enzalutamide.

### [Item 49]

The method of item 47 or 48, wherein the androgen synthesis inhibitor is abiraterone.

### [Item 50]

A method of treating cancer that exhibits resistance to androgen deprivation therapy, comprising administering alvocidib or a pharmaceutically acceptable salt thereof.

### [Item 51]

Use of alvocidib or a pharmaceutically acceptable salt thereof for the manufacture of a drug for treating cancer that exhibits resistance to androgen deprivation therapy.

### [Item 52]

Alvocidib or a pharmaceutically acceptable salt thereof for use in the treatment of cancer that exhibits resistance to androgen deprivation therapy.

### [Item 53]

A method of diagnosing whether a subject is suited to therapy using alvocidib or a pharmaceutically acceptable salt thereof, comprising measuring phosphorylation of an androgen receptor of the subject.

### [Item 54]

A kit for use in diagnosing whether a subject is suited to therapy using alvocidib or a pharmaceutically acceptable salt thereof, comprising means for measuring phosphorylation of an androgen receptor of the subject.

### [Item 55]

A cell having androgen non-dependent cell proliferation, wherein the cell is enzalutamide resistant with expression of AR-V7.

### [Item 56]

A method of screening for cells with an androgen non-dependent proliferation capability, comprising:

culturing an androgen dependent cell line;

maintaining long-term cell culture while repeatedly exchanging a medium; and

separating a cell clone that has acquired a proliferation capability and culturing the cell clone.

The present invention is intended so that one or more of the aforementioned features can be provided not only as the explicitly disclosed combinations, but also as other combinations thereof. Additional embodiments and advantages of the present invention are recognized by those skilled in the art by reading and understanding the following detailed description as needed.

### Advantageous Effects of Invention

The alvocidib of the invention or a pharmaceutically acceptable salt thereof is effective in the treatment and/or prevention of cancer that exhibits therapeutic resistance to an androgen receptor antagonist and/or an androgen synthesis inhibitor, and is particularly effective in the treatment and/or prevention of prostate cancer expressing a mutant androgen receptor.

Further, a patient for whom alvocidib or a pharmaceutically acceptable salt thereof is effective can be predicted by measuring the phosphorylation status of an androgen receptor of a cancer cell from a subject based on the present invention.

### BRIEF DESCRIPTION OF DRAWINGS

FIG. 1 is a diagram measuring the amount of PSA production of each cell by immunoblotting.

FIG. 2 is a phase contrast microscope image of an AILNCaP14 cell line and AILNCaP15 cell line cultured under androgen deprivation.

FIG. 3 is a diagram identifying the expression of mRNA of AR and AR-V7 of an LNCaP cell line, AILNCaP4 cell line, AILNCaP7 cell line, AILNCaP14 cell line, and AILNCaP15 cell line using agarose gel electrophoresis.

FIG. 4 is a diagram showing the effect of suppressing cell proliferation of enzalutamide on an LNCaP cell line, AILNCaP14 cell line, and AILNCaP15 cell line.

FIG. 5 is a diagram showing the effect of suppressing cell proliferation of alvocidib on an LNCaP cell line, AILNCaP14 cell line, and AILNCaP15 cell line.

FIG. 6 is a diagram measuring the effect of suppressing AR phosphorylation over time of alvocidib on an AILNCaP14 cell line and AILNCaP15 cell line by immunoblotting.

FIG. 7 is a diagram showing a result of immunofluorescent staining of AR in an alvocidib administered group and unadministered group in an AILNCaP14 cell line.

FIG. 8 is a diagram measuring the effect of suppressing AR phosphorylation of voruciclib on an AILNCaP14 cell line by immunoblotting.

FIG. 9 is a diagram showing a summary of a test scheme of a tumor proliferation suppression test on AILNCaP14 cell derived subcutaneous xenograft mice.

FIG. 10 is a diagram showing the ratio of increase in tumor volume in the alvocidib administered group and negative control group in a tumor proliferation suppression test on AILNCaP14 cell derived subcutaneous xenograft mice.

### DESCRIPTION OF EMBODIMENTS

The present invention is described hereinafter in more detail. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. The terms used herein should also be understood as being used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

Cyclin-dependent kinase (CDK) is an important regulatory factor that modulates the progression of a cell cycle or the like, so that a selective CDK inhibitor would be a useful chemotherapeutic agent. Alvocidib, roniciclib, dinaciclib, and voruciclib are known as representative CDK inhibitors.

Alvocidib (Flavopiridol, chemical name: 2-(2-chlorophenyl)-5,7-dihydroxy-8-[(3S,4R)-3-hydroxy-1-methylpiperidin-4-yl]-4H-1-benzopyran-4-one) is a synthetic flavone having the following structure:

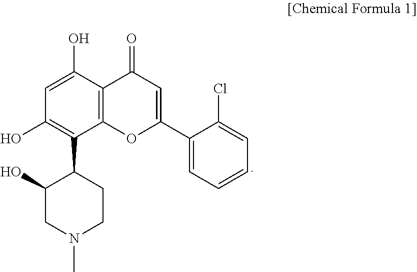 [SMILES: CN1CC[C@H](c2c(O)cc(O)c3c(=O)cc(-c4ccccc4Cl)oc23)[C@H](O)C1]

Alvocidib is a potent and selective inhibitor of CDK, which has antitumor activity against various tumor cell systems such as human lung cancer and breast cancer and inhibits tumor growth in a xenograft model. Alvocidib inhibits polymerase II driven transcription through CDK9 inhibition. Treatment with alvocidib inhibits CDK9, which forms a part of a complex known as a positive transcription elongation factor or P-TEFb and reduces the expression of important cancer genes such as MYC and expression of important anti-apoptosis proteins such as MCL1. Therefore, alvocidib is an appealing therapeutic agent for cancer. Currently, clinical development thereof for use in hematological cancer is ongoing.

“Alvocidib” herein can be in a form of a hydrate and/or solvate. Thus, hydrates and/or solvates of “alvocidib or a pharmaceutically acceptable salt thereof” are also encompassed by the compound of the invention.

Alvocidib herein can be appropriately used as a pharmaceutically acceptable salt thereof.

“Pharmaceutically acceptable salt” refers to a salt prepared from a pharmaceutically nontoxic acid (including inorganic acids and organic acids). Examples of pharmaceutically acceptable salts include, but are not limited to, acetic acid salt, alginic acid salt, anthranilic acid salt, benzenesulfonic acid salt, benzoic acid salt, camphorsulfonic acid salt, citric acid salt, ethenesulfonic acid salt, formic acid salt, fumaric acid salt, gluconic acid salt, glutamic acid salt, glucorenic acid salt, galacturonic acid salt, glycidic acid salt, hydrobromic acid salt, hydrochloric acid salt, isethionic acid salt, lactic acid salt, maleic acid salt, malic acid salt, mandelic acid salt, methanesulfonic acid salt, mucic acid salt, nitric acid salt, pamoic acid salt, pantothenic acid salt, phenylacetic acid salt, propionic acid salt, phosphoric acid salt, salicylic acid salt, stearic acid salt, succinic acid salt, sulfanilic acid salt, sulfuric acid salt, tartaric acid salt, p-toluenesulfonic acid salt, and the like. Preferred examples of pharmaceutically acceptable salts include hydrobromic acid salt and hydrochloric acid salt. The most preferred examples of pharmaceutically acceptable salts include hydrochloric acid salt.

“Alvocidib or a pharmaceutically acceptable salt thereof” obtained as a crystal may have a crystalline polymorphism. The “alvocidib or a pharmaceutically acceptable salt thereof” herein encompasses any crystalline form.

“Alvocidib or a pharmaceutically acceptable salt thereof” may have one or optionally more asymmetric carbon atoms and may have geometrical isomerism or axial chilarity, so that alvocidib or a pharmaceutically acceptable salt thereof may be present as several types of stereoisomers. In the present invention, such stereoisomers and mixtures and racemates thereof are also encompassed by the compound of the invention.

Deuterism converted forms produced from converting any one or more ¹H of “alvocidib or a pharmaceutically acceptable salt thereof” to ²H(D) are also encompassed by the compound of the invention.

“Androgen receptor antagonist” herein refers to an agent that inhibits androgen from binding to an androgen receptor, including CRPC as an applicable disease.

Examples of “androgen receptor antagonist” include enzalutamide, apalutamide, darolutamide, and the like. Preferred examples of “androgen receptor antagonist” include enzalutamide.

“Androgen synthesis inhibitor” herein refers to an agent that inhibits biosynthesis of androgen. “Androgen synthesis inhibitor” is preferably an agent that has action to inhibit conversion of progesterone to androgen by selectively inhibiting CYP17. Specific examples of “androgen synthesis inhibitor” include abiraterone, galeterone, and the like.

A wild-type androgen receptor (wild-type AR) refers to a basic phenotype of an androgen receptor found in normal cells. As used herein, wild-type androgen receptor is synonymous with normal androgen receptor (normal AR), referring to an androgen receptor without a mutation. When simply denoted as androgen receptor (AR), this refers to a wild-type androgen receptor (wild-type AR).

A mutant androgen receptor (mutant AR) refers to an androgen receptor mutated from a wild-type androgen receptor. Examples of mutations in mutant androgen receptors include “mutation due to a splicing variant”, “point mutation”, and “mutation due to post-translational processing”. Examples of “point mutation” include “mutation due to an amino acid substitution”, “mutation due to an amino acid deletion”, and “mutation due to an amino acid insertion”. Examples of mutant androgen receptors thus include “splicing variant”, “mutant androgen receptor due to a point mutation”, and “mutant androgen receptor due to post-translational processing”.

Specific examples of “splicing variant” include AR-V1 (androgen receptor-variant 1), AR-V2 (androgen receptor-variant 2), AR-V3 (androgen receptor-variant 3), AR-V4 (androgen receptor-variant 4), AR-V5 (androgen receptor-variant 5), AR-V567es (androgen receptor-variant 567es), AR-V6 (androgen receptor-variant 6), AR-V7 (androgen receptor-variant 7), and AR-V12 (androgen receptor-variant 12).

Preferred examples of “splicing variant” include AR-V7, AR-V12, and AR-V567es.

Specific examples of more preferred “splicing variant” include AR-V7.

Specific examples of “mutant androgen receptor due to a point mutation” include T877A (T878A), D879G (D878G), W741C, W741L, M749L, R629Q, G142V, P533S, T575A, H874Y, and F876L.

Specific examples of preferred “mutant androgen receptor due to a point mutation” include F876L. Apalutamide and enzalutamide that exhibit antagonistic activity against normal androgen receptors have a feature of acting as an agonist against mutant androgen receptor due to a point mutation F876L.

“Anti-androgen receptor antibody” refers to an antibody that specifically recognizes an androgen receptor. “Anti-androgen receptor antibody” includes antibodies that recognize a wild-type androgen receptor, as well as antibodies that specifically recognize an androgen receptor with phosphorylation of a specific residue. Specific examples of anti-androgen receptor antibodies include anti-AR antibodies (Santa Cruz Biotechnology Inc., Cat no. sc-816), anti-pAR^{ser81} antibodies (Merck, Cat no. 04-078), and anti-pAR^{ser210+213} antibodies (Abcam, Cat no. ab45089).

“Cancer” herein refers to malignant tumor.

Examples of “cancer” include acute leukemia, chronic lymphocytic leukemia, chronic myelogenous leukemia, polycythemia vera, malignant lymphoma, plasma cell tumor, multiple myeloma, myelodysplastic syndrome, brain tumor, head and neck cancer, esophageal cancer, thyroid cancer, small cell lung cancer, non-small cell lung cancer, thymoma/thymic carcinoma, breast cancer, gastric cancer, gallbladder/bile duct cancer, liver cancer, hepatocellular carcinoma, pancreatic cancer, colon cancer, rectal cancer, anal cancer, gastrointestinal stromal tumor, choriocarcinoma, endometrial cancer, cervical cancer, ovarian cancer, bladder cancer, prostate cancer, urothelial cancer, renal cancer, renal cell cancer, testicular tumor, testicular germ cell tumor, ovarian germ cell tumor, Wilms tumor, skin cancer, malignant melanoma, neuroblastoma, osteosarcoma, Ewing sarcoma, and soft tissue sarcoma. Preferred examples include cancer in a state exhibiting resistance to androgen deprivation therapy.

Preferred examples of “cancer” include prostate cancer, breast cancer, ovarian cancer, and bladder cancer.

More preferred examples of “cancer” include prostate cancer.

Another preferred embodiment as “cancer” includes “cancer that exhibits therapeutic resistance to an androgen receptor antagonist and/or an androgen synthesis inhibitor”.

Another more preferred embodiment as “cancer” includes cancer that exhibits therapeutic resistance to enzalutamide.

Another more preferred embodiment as “cancer” includes cancer that exhibits therapeutic resistance to abiraterone.

Still another preferred embodiment as “cancer” includes “cancer expressing a mutant androgen receptor”.

Examples of “prostate cancer” herein include “castration resistant prostate cancer (CRPC)”, more preferably “castration resistant prostate cancer that exhibits therapeutic resistance to an androgen receptor antagonist and/or an androgen synthesis inhibitor”, and still more preferably “castration resistant prostate cancer that exhibits therapeutic resistance to enzalutamide, apalutamide, and/or abiraterone”.

Another preferred embodiment as “prostate cancer” includes “castration resistant prostate cancer with elevated phosphorylation of serine 81, and serine 210 or serine 213 of an androgen receptor”. Phosphorylation of an androgen receptor can be measured by, for example, the method described in the following Example 3.

The “numerical value” described after serine in serine 81, serine 210, and serine 213 refers to the residue number in the amino acid sequence of an androgen receptor. For example, pAR^{ser81} refers to an androgen receptor with phosphorylation of the 81^{st} serine residue of the androgen receptor. pAR^{ser210+213} refers to an androgen receptor with phosphorylation of the 210^{th} or 213^{th} serine residue. 210+213 is denoted in this manner because the 210^{th} serine residue in one genetic polymorphism would be 213^{th} in another genetic polymorphism due to a discrepancy in the sequences that occurs in genetic polymorphisms found in wild-type androgen receptors. This does not refer to an androgen receptor with phosphorylation of two serine residues.

“Castration resistant prostate cancer (CRPC)” is also known as hormone-refractory prostate cancer. This indicates prostate cancer in a state exhibiting resistance to androgen deprivation therapy. Androgen deprivation therapy is a method of treating cancer that exhibits androgen dependence for suppressing production or secretion of androgen and blocking androgen from binding to an androgen receptor. For example, surgical castration, as well as administration of an LH-RH agonist (goserelin, leuprorelin, or the like), flutamide, bicalutamide, nilutamide, or the like is performed against prostate cancer in order to suppress androgen secretion from the testes. See Clinical Practice Guideline for Prostate Cancer 2016, Edited by The Japanese Urological Association, Medical Review Co., Ltd. This document has the following description regarding castration resistant prostate cancer: 2012 version of “Clinical Practice Guideline for Prostate Cancer” describes that progression in the pathological condition from the viewpoint of post-hormonal therapy PSA value is “defined as a PSA value measured after 4 weeks or longer of 25% or greater from the minimum value, and an increase of 2.0 ng/mL or greater” in accordance with the “General Rule for Clinical and Pathological Studies on Prostate Cancer” (2010). With regard to PSA values and definition of CRPC in the recent American and European guidelines, the European Association of Urology (EAU) guidelines define it as serum testosterone value of less than 50 ng/dL and (1) 3 consecutive increases in PSA value at measurement intervals of 1 week or longer and an increase of 50% or more from the minimum value observed twice, and a PSA value of 2.0 ng/mL or greater, or (2) exacerbation or manifestation of a new lesion on an image. The standard for pathological progression from the viewpoint of PSA values in Prostate Cancer Working Group 2 (PCWG2) (2008) is defined as consecutive increases in PSA value at a measurement interval of 1 week or longer, and a PSA value of 2.0 ng/mL or greater. The PSA value was changed to 1.0 ng/mL in PCWG3 (2015).

“Cancer that exhibits resistance to androgen deprivation therapy” indicates cancer exhibiting androgen dependency, which has acquired resistance after administering androgen deprivation therapy for a certain period of time. Cancer is determined herein as cancer that exhibits resistance to androgen deprivation therapy when the value of cancer specific antigen (e.g., PSA value) measured after 4 weeks or longer is 25% or greater from the minimum value and the increase is equal to or greater than a specific value (e.g., 2.0 ng/mL) in light of the standards specified in the aforementioned Clinical Practice Guideline for Prostate Cancer 2016. Cancer is determined as exhibiting resistance herein when a PSA value is 25% or greater from the minimum value and an increase is 2.0 ng/mL or greater, unless specifically noted otherwise.

“Cancer that exhibits therapeutic resistance to an androgen receptor antagonist” indicates cancer exhibiting androgen dependency, which has acquired resistance after a therapy with an androgen receptor antagonist for a certain period of time. Cancer is determined herein as cancer that exhibits therapeutic resistance to an androgen receptor antagonist when the value of cancer specific antigen (e.g., PSA value) measured after 4 weeks or longer is 25% or greater from the minimum value and the increase is equal to or greater than a specific value (e.g., 2.0 ng/mL) in light of the standards specified in the aforementioned Clinical Practice Guideline for Prostate Cancer 2016. Cancer is determined as exhibiting resistance herein when a PSA value is 25% or greater from the minimum value and an increase is 2.0 ng/mL or greater, unless specifically noted otherwise.

“Cancer that exhibits therapeutic resistance to an androgen synthesis inhibitor” indicates cancer exhibiting androgen dependency, which has acquired resistance after a therapy with an androgen synthesis inhibitor for a certain period of time. Cancer is determined herein as cancer that exhibits therapeutic resistance to an androgen synthesis inhibitor when the value of cancer specific antigen (e.g., PSA value) measured after 4 weeks or longer is 25% or greater from the minimum value and the increase is equal to or greater than a specific value (e.g., 2.0 ng/mL) in light of the standards specified in the aforementioned Clinical Practice Guideline for Prostate Cancer 2016. Cancer is determined as exhibiting resistance herein when a PSA value is 25% or greater from the minimum value and an increase is 2.0 ng/mL or greater, unless specifically noted otherwise.

“Patient with a serum testosterone concentration reduced to a castration level by castration and/or drug therapy” indicates a patient with a serum testosterone concentration reduced to castration level (0.5 ng/mL or less) by castration and/or drug therapy.

“Prevention” herein is an act of administering the active ingredient of the invention to an individual who has not developed a target disease. For example, prevention is intended to prevent the development of a disease.

“Therapy (treatment)” herein is an act of administering the active ingredient of the invention to an individual (patient) diagnosed to have developed a disease by a physician. For example, therapy is intended to alleviate a disease or symptom, prevent increase in cancer or tumor, or restore the condition to that prior to developing the disease. Even when the objective of administration is prevention of exacerbation of a disease or symptom or prevention of increase in cancer or tumor, this is an act of therapy if administered to a patient.

When administering the compound of the invention, the amount used varies depending on the symptom, age, administration method, or the like, but an effect is expected by administering 0.01 mg (preferably 0.1 mg) as the lower limit to 1000 mg (preferably 100 mg) as the upper limit per day for an adult, separated into one or several doses depending on the symptom for intravenous injection. Examples of the dosing schedule thereof include a single dose, once daily administration for 3 consecutive days, twice daily administration for 7 consecutive days, and the like. Each of the administration methods described above can also be repeated with an interval of about 1 day to about 60 days.

The compound of the invention can be administered via parenteral administration or oral administration, but is preferably administered by a parenteral method, and more preferably by an intravenous injection.

Cancer can be more effectively prevented or treated by combining (1) administration of an effective amount of the compound of the invention, with 1 to 3 selected from the group consisting of (2) (i) administration of an effective amount of another anticancer agent, (ii) administration of an effective amount of a hormonal therapeutic agent, and (iii) a non-drug therapy. Examples of non-drug therapies include surgery, radiation therapy, gene therapy, thermotherapy, cryotherapy, laser therapy, and the like. Two or more thereof can also be combined.

The compound of the invention can be used concomitantly with another drug in order to enhance the effect thereof. Specifically, the compound of the invention can be used concomitantly with a drug such as a hormonal therapy agent, a chemotherapeutic agent, an immunotherapeutic agent, or an agent inhibiting a cell growth factor and its receptor action. A drug that can be used concomitantly with the compound of the invention is abbreviated as concomitantly used drug hereinafter.

While the compound of the invention exhibits an excellent anticancer action when used as a single agent, the effect can be further enhanced or the QOL of a patient can be improved by concomitantly using one or several of the concomitantly used drugs (concomitant use of multiple agents).

Examples of “hormonal therapeutic agent” include fosfestrol, diethylstilbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, tadenan, mepartricin, raloxifene, ormeroxifene, levormeloxifene, antiestrogens (e.g., tamoxifen citrate, toremifene citrate, and the like), pill formulations, mepitiostane, testolactone, aminoglutethimide, LH-RH derivatives (LH-RH agonists (e.g., goserelin acetate, buserelin, leuprorelin, and the like) and LH-RH antagonists), droloxifene, epitiostanol, ethinylestradiol sulfonate, aromatase inhibitors (e.g., fadrozole hydrochloride, anastrozole, letrozole, exemestane, vorozole, formestane, and the like), flutamide, bicalutamide, nilutamide, androgen receptor antagonists (e.g., apalutamide and enzalutamide), androgen synthesis inhibitors (e.g., abiraterone and the like), adrenocortical hormone based agents (e.g., dexamethasone, prednisolone, betamethasone, triamcinolone, and the like), retinoids, agents that slow the metabolism of retinoids (e.g., liarozole and the like), and the like.

For example, an alkylating agent, antimetabolite, anticancer antibiotic, plant derived anticancer agent, molecularly targeted therapeutic agent, immunomodulator, other chemotherapeutic agent, or the like can be used as a “chemotherapeutic agent”. Representative examples thereof are described below.

Examples of “alkylating agents” include nitrogen mustard, nitrogen mustard N-oxide hydrochloride, chlorambucil, cyclophosphamide, ifosfamide, thiotepa, carboquone, improsulfan tosylate, busulfan, nimustine hydrochloride, mitobronitol, melphalan, dacarbazine, ranimustine, estramustine phosphate sodium, triethylenemelamine, carmustine, lomustine, streptozocin, pipobroman, etoglucide, carboplatin, cisplatin, miboplatin, nedaplatin, oxaliplatin, altretamine, ambamustine, dibrospidium hydrochloride, fotemustine, prednimustine, pumitepa, ribomustin, temozolomide, thiotepa, treosulfan, trofosfamide, zinostatin stimalamer, adozelesin, cystemustine, bizelesin, DDS formulations thereof, and the like.

Examples of “antimetabolite” include mercaptopurine, 6-mercaptopurine riboside, thioinosine, methotrexate, pemetrexed, enocitabine, cytarabine, cytarabine ocfosfate, ancitabine hydrochloride, 5-FU based agents (e.g., fluorouracil, tegafur, UFT, doxifluridine, carmofur, galocitabine, emitefur, capecitabine, and the like), aminopterin, nelzarabine, leucovorin calcium, tabloid, butocin, calcium folinate, calcium levofolinate, cladribine, emitefur, fludarabine, gemcitabine, hydroxycarbamide, pentostatin, piritrexim, idoxuridine, mitoguazone, tiazofurin, ambamustine, bendamustine, DDS formulations thereof, and the like.

Examples of “anticancer antibiotic” include actinomycin D, actinomycin C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarkomycin, carzinophilin, mitotane, zorubicin hydrochloride, mitoxantrone hydrochloride, idarubicin hydrochloride, DDS formulations thereof, and the like.

Examples of “plant derived anticancer agent” include etoposide, etoposide phosphate, vinblastine sulfate, vincristine sulfate, vindesine sulfate, teniposide, paclitaxel, docetaxel, DJ-927, vinorelbine, irinotecan, topotecan, DDS formulations thereof, and the like.

Examples of “molecularly targeted therapeutic agent” include imatinib, gefitinib, erlotinib, sorafenib, dasatinib, sunitinib, nilotinib, lapatinib, pazopanib, ruxolitinib, crizotinib, vemurafenib, vandetanib, ponatinib, cabozantinib, tofacitinib, regorafenib, bosutinib, axitinib, dabrafenib, trametinib, nintedanib, idelalisib, ceritinib, lenvatinib, palbociclib, alectinib, afatinib, osimertinib, ribociclib, abemaciclib, brigatinib, neratinib, copanlisib, cobimetinib, ibrutinib, acalabrutinib, encorafenib, binimetinib, baricitinib, fostamatinib, lorlatinib, erdafitinib, entrectinib, dacomitinib, sirolimus, everolimus, temsirolimus, olaparib, rucaparib, niraparib, venetoclax, azacitidine, decitabine, vorinostat, panobinostat, romidepsin, bortezomib, carfilzomib, ixazomib, and the like.

Examples of “immunomodulator” include lenalidomide, pomalidomide, and the like.

Examples of “other chemotherapeutic agent” include sobuzoxane and the like.

Examples of “immunotherapeutic agent (BRM)” include picibanil, krestin, sizofiran, lentinan, ubenimex, interferon, interleukin, macrophage colony stimulating factor, granulocyte-colony stimulating factor, erythropoietin, lymphotoxin, BCG vaccine, Corynebacterium parvum, levamisole, polysaccharide K, procodazole, anti-CTLA4 antibody, anti-PD-1 antibody, anti-PD-L1 antibody, and Toll-like Receptor agonist (e.g., TLR7 agonist, TLR8 agonist, TLR9 agonist, and the like).

The cell growth factor in an agent inhibiting a cell growth factor and its receptor action can be any substance, as long as it is a substance promoting cell proliferation. A cell growth factor is generally a peptide having a molecular weight of 20,000 or less and exerting action at a low concentration by binding with a receptor. Specific examples thereof include EGF (epidermal growth factor) or substances having substantially the same activity as EGF (e.g., TGF-alpha and the like), insulin or substances having substantially the same activity as insulin (e.g., insulin, IGF (insulin-like growth factor)-1, IGF-2, and the like), FGF (fibroblast growth factor) or substances having substantially the same activity as FGF (e.g., acidic FGF, basic FGF, KGK (keratinocyte growth factor), FGF-10, and the like), and other cell growth factors (e.g., CSF (colony stimulating factor), EPO (erythropoietin), IL-2 (interleukin-2), NGF (nerve growth factor), PDGF (platelet-derived growth factor), TGF-beta (transforming growth factor beta), HGF (hepatocyte growth factor), VEGF (vascular endothelial growth factor), heregulin, angiopoietin, and the like).

The dosing period of the compound of the invention and a concomitantly used drug is not limited. They can be administered simultaneously or differentially to a target of administration. The compound of the invention and a concomitantly used drug can also be prepared as a combined drug. The amount of concomitantly used drug to be administered can be appropriately selected based on clinically used doses. The blend ratio of the compound of the invention to a concomitantly used drug can be appropriately selected depending on the subject of administration, route of administration, target disease, symptom, combination, or the like. If, for example, the subject of administration is a human, 0.01 to 100 parts by weight of concomitantly used drug can be used with respect to 1 part by weight of the compound of the invention. They can also be used in combination with an agent (concomitantly used drug) such as an antiemetic, sleep inducing agent, or anticonvulsive in order to suppress side effects thereof.

The present invention is described in detail while providing Examples hereinafter, but the present invention is not limited thereto in any manner.

### EXAMPLES

### Example 1

Novel cell line models (AILNCaP14 cell line and AILNCaP15 cell line) which reflect the pathological condition of enzalutamide resistant CRPC accompanied with expression of AR-V7 were established through the following Examples 1-1 to 1-5.

### Example 1-1

Obtaining Clones that have Acquired Androgen Non-Dependent Cell Proliferation Capability

An RPMI 1640 medium containing 10% fetal bovine serum (FBS) was dispensed into a p100 cell culture plate. 1×10⁶ cells of “androgen dependent prostate cancer cell line (LNCaP cell line, obtained from American Type Culture Collection (ATCC))” were seeded and cultured for 48 hours in the presence of 5% CO₂ at 37° C. After the culturing, the medium was removed from the plate. The cultured cells were washed twice with phosphate buffer (PBS), and then continued to be cultured over 3 months in a phenol red free RPMI 1640 medium containing activated carbon-treated 10% fetal bovine serum (csFBS) while exchanging the medium every 4 to 5 days in the presence of 5% CO₂ at 37° C. Although most cells die under this culture condition due to apoptosis, a plurality of cell clones that have acquired proliferation capability were formed at 3 months from the start of culture. These clones were individually transferred onto a 48-well plate, and culture was continued under the same culture condition. After transferring the cell clones that reached 80% confluence from this culture to a 24-well plate and culturing, the clones were ultimately maintained and cultured in a 6-well plate.

A plurality of clones that have acquired an androgen non-dependent cell proliferation capability were obtained by the culturing method described above.

### Example 1-2

Measurement of PSA Production by Immunoblotting

The amount of PSA production was measured for 5 of the clones obtained in Example 1-1. The LNCaP cell line described in Example 1-1 was used as the control.

Cells were cultured for 180 days in a phenol red free RPMI 1640 medium containing activated carbon-treated 10% fetal bovine serum (csFBS) in the presence of 5% CO₂ at 37° C. in the same manner as Example 1-1. After the culturing, each of 1×10⁶ cells were washed with ice cooled PBS, and then treated with cell lysis buffer containing 8 M of urea, 20 mM of Tris hydrochloride (pH 7.4), 1 mM of EDTA, and protease inhibitor (Nacalai tesque, Cat no. 03969) and phosphatase inhibitor (Nacalai tesque, Cat no. 07575-51)—added 1.0% Triton X. The cell lysate was then subjected to high speed centrifugation (21,500 G) for 20 minutes at 4° C. The total amount of cell protein was quantified using a protein quantification reagent (Thermo Fisher Scientific, Cat no. 23227). The cell lysate was separated by SDS-PAGE and transcribed onto a nitrocellulose membrane (Merck Millipore, Cat no. PVH304F0), and then chemiluminescence from immunoblotting using an HRP (Horseradish peroxidase) labeled secondary antibody was detected using an anti-PSA antibody (Santa Cruz Biotechnology Inc., Cat no. sc-7638) and anti-beta actin antibody (Wako, Cat no. 013-24553) as primary antibodies to measure the amount of PSA production.

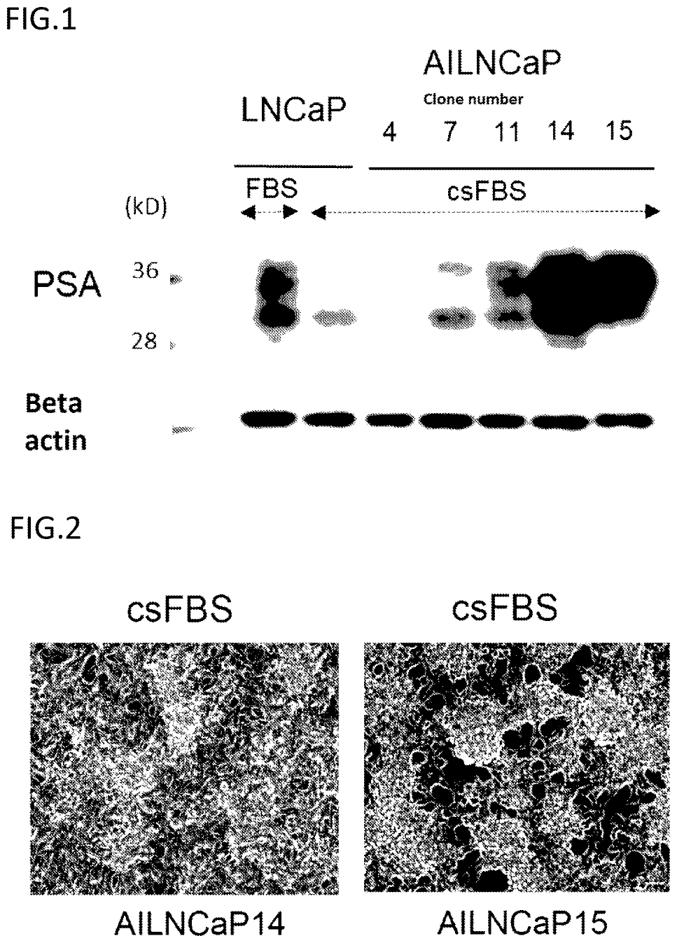

FIG. 1 shows the result of measuring the amount of PSA production. High PSA production was observed in the cell lines of clone numbers 14 and 15 compared to LNCaP cells even under androgen deprivation (csFBS) in view of the aforementioned measurement results. This result is a reflection of CRPC in actual clinical setting. Hereinafter, these cell lines are called “AILNCaP14 cell line” and “AILNCaP15 cell line”, respectively.

### Example 1-3

Study of Proliferation Capability of AILNCaP14 Cell Line and AILNCaP15 Cell Line Under Androgen Deprivation

2.5×10⁶ cells/well of AILNCaP14 cell line and AILNCaP15 cell line were cultured for 6 days in a phenol red free RPMI 1640 medium containing activated carbon-treated 10% fetal bovine serum (csFBS) in the presence of 5% CO₂ at 37° C. in the same manner as Example 1-1. After the culturing, the cells were captured under phase contrast conditions with Keyence's BZ-9000 series (BIOREVO).

FIG. 2 shows the resulting phase contrast microscope pictures. The cell count after the culture was 5×10⁶ cells/well for both cell lines, increasing to 2-fold compared to before culture. This result confirms that the AILNCaP14 cell line and AILNCaP15 cell line proliferate well under androgen deprivation.

### Example 1-4

Identification of Mutant AR Gene of AILNCaP4 Cell Line, AILNCaP7 Cell Line, AILNCaP14 Cell Line, and AILNCaP15 Cell Line

Expression of mRNA of AR and AR-V7 splicing variant was identified by reverse transcription PCR and agarose gel electrophoresis.

2.5×10⁶ cells/well of AILNCaP14 cell line and AILNCaP15 cell line were seeded on a 6-well plate to which 10 mL/well of medium was dispensed. After culturing for 6 days, total RNA was extracted from cultured cells using RNeasy® Mini Kit (QIAGEN Cat no. 74106). 2 μg of total RNA was used to synthesize cDNA by using ReverTra ACE qPCR RT Master Mix (TOYOBO Code no. FSQ-201) in accordance with the protocol thereof. PCR amplicons were identified by agarose gel electrophoresis.

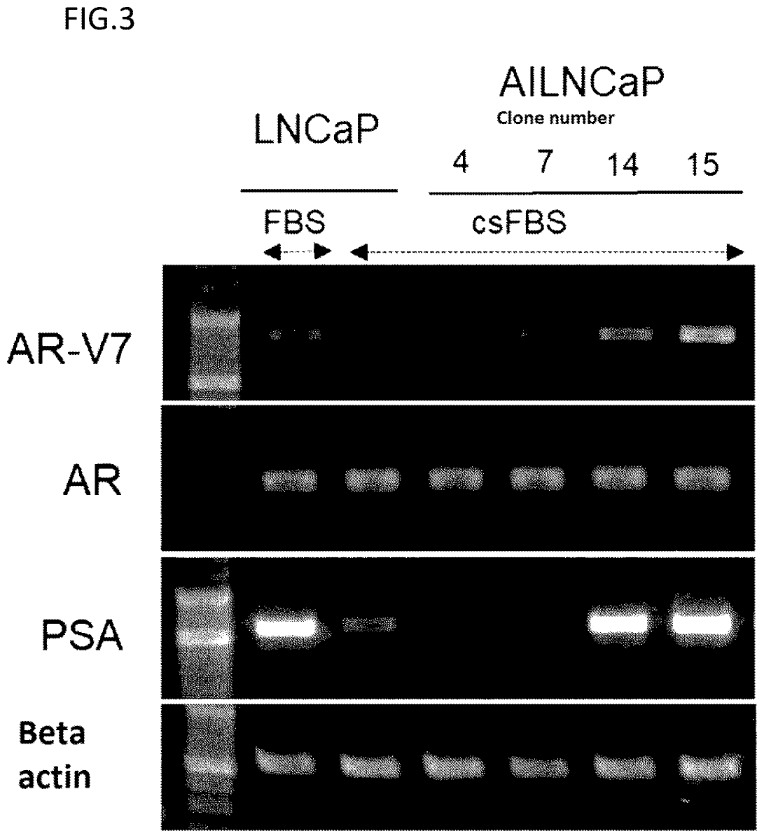

Table 1 shows the sequences of primers used in reverse transcription PCR. Table 2 shows the reverse transcription PCR conditions. FIG. 3 shows the result.

TABLE 1
Primer sequences used in reverse transcription PCR

| | | |
|---|---|---|
| AR | Forward primer | CCATCTTGTCGTCTTCGGAAATGTTATGAAGC (SEQ ID NO: 1) |
| | Reverse primer | AGCTTCTGGGTTGTCTCCTCAGTGG (SEQ ID NO: 2) |
| AR-V7 | Forward primer | TGTCACTATGGAGCTCTCACATGTGG (SEQ ID NO: 3) |
| | Reverse primer | CTGTGGATCAGCTACTACCTTCAGCTC (SEQ ID NO: 4) |
| Beta actin | Forward primer | TCTACAATGAGCTGCGTGTG (SEQ ID NO: 5) |
| | Reverse primer | AGCACACAGCATGAACTTGG (SEQ ID NO: 6) |

TABLE 2
Reverse transcription PCR conditions

| | | | |
|---|---|---|---|
| AR | 94° C. | 2 minutes | — |
| | 94° C. | 30 seconds | 23 cycles |
| | 50° C. | 30 seconds | |
| | 68° C. | 30 seconds | |
| | 68° C. | 5 minutes | — |
| AR-V7 | 94° C. | 2 minutes | — |
| | 94° C. | 30 seconds | 28 cycles |
| | 63° C. | 30 seconds | |
| | 68° C. | 30 seconds | |
| | 68° C. | 5 minutes | — |
| Beta actin | 94° C. | 2 minutes | — |
| | 94° C. | 30 seconds | 17 cycles |
| | 50° C. | 30 seconds | |
| | 68° C. | 30 seconds | |
| | 68° C. | 5 minutes | — |

In view of the results of Example 1-4, “LNCaP cell line” was found to express mRNA of wild-type AR under androgen deprivation, but expression of mRNA of a splicing variant AR-V7 was hardly found. Meanwhile, it was confirmed that expression of mRNA of a splicing variant AR-V7 was found in addition to expression of mRNA of wild-type AR in the “AILNCaP14 cell line” and “AILNCaP15 cell line”.

### Example 1-5

Cell Proliferation Suppression Test Using Enzalutamide

5×10³ cells of the LNCaP cell line, 4×10⁴ cells of the AILNCaP14 cell line, and 6×10⁴ cells of the AILNCaP15 cell line were each seeded on a 24-well plate to which 1 mL/well of medium was dispensed. After 72 hours of culture under the conditions of 5% CO₂ at 37° C., a DMSO solution of enzalutamide was diluted with a medium so that the final concentrations of enzalutamide would be 0.625, 1.25, 2.5, and 5 uM and added to each well (3 well/concentration, final volume of 2 mL) for each plate. As a control, enzalutamide-free DMSO was similarly diluted with a medium and added. After culturing each cell line for an additional two weeks, the status of cell proliferation was measured by cell staining (Bommi-Reddy et al. Proc Natl Acad Sci USA. 105: 16484-16489 (2008)) using crystal violet.

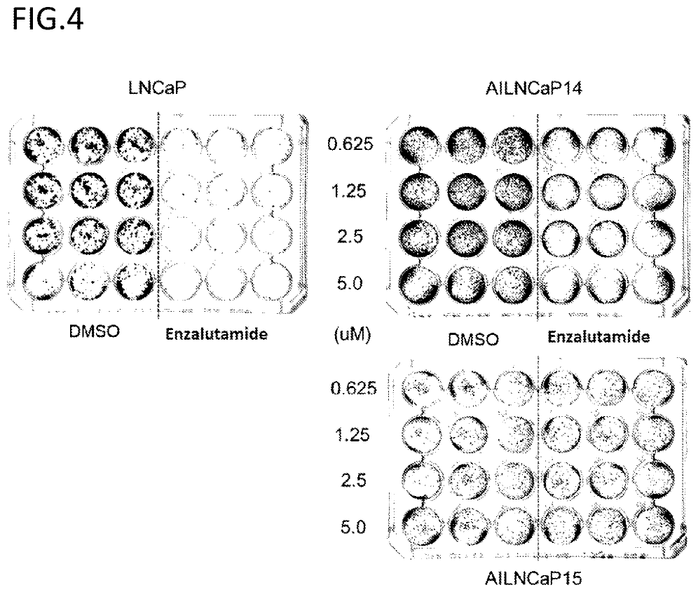

FIG. 4 shows the results of Example 1-5. From the test results of Example 1-5, a clear suppression of cell proliferation was observed upon addition of enzalutamide at a final concentration of 0.625 uM or greater in the LNCaP cell line, whereas significant weaker suppression of cell proliferation compared to the LNCaP cell line was confirmed at a final concentration of 5 uM in the AILNCaP14 cell line and the AILNCaP15 cell line (IC₅₀ >5 uM). Thus, the AILNCaP14 cell line and the AILNCaP15 cell line were found to have acquired resistance to enzalutamide compared to the LNCaP cell line.

The “AILNCaP14 cell line” and the “AILNCaP15 cell line” isolated in Examples 1-1 and 1-2 had high expression of PSA and proliferated well under androgen deprivation (Example 1-3). Furthermore, said cell lines were found to express androgen receptor splicing variant AR-V7 (Example 1-4) and have acquired resistance to enzalutamide. In view of the above, “AILNCaP14 cell line” and “AILNCaP15 cell line” are cell line models that beneficially reflect the pathological condition of castration resistant prostate cancer that exhibits therapeutic resistance to an androgen receptor antagonist and/or an androgen synthesis inhibitor.

### Example 2

Cell Proliferation Suppression Test Using Alvocidib (Flavopiridol)

Alvocidib hydrochloride was used in place of enzalutamide to study the effect of suppressing cell proliferation of alvocidib on AILNCaP14 cell line and AILNCaP15 cell line by the same experimental procedure as Example 1-5. The final concentrations of alvocidib were set to be 0.125, 0.25, 0.5, and 1.0 uM.

FIG. 5 shows the results of Example 2. It was confirmed that a very potent effect of suppressing cell proliferation was observed for both types of cells by allowing alvocidib at a final concentration of 0.25 uM or greater to act on the AILNCaP14 cell line and AILNCaP15 cell line (IC₅₀=0.125 uM to 0.25 uM). This result shows a significant and different effect in that suppression of cell proliferation was exhibited from a low concentration against the AILNCaP14 cell line and AILNCaP15 cell line, on which an effect of suppressing cell proliferation is hardly observed even with enzalutamide used in the treatment of castration resistant prostate cancer.

### Example 3

AR Phosphorylation Suppression Test Using Alvocidib

After seeding 2.5×10⁶ cells/well of the AILNCaP14 cell line and AILNCaP15 cell line on a 6-well plate to which 10 mL/well of medium was dispensed and culturing the cells for 6 days under the conditions of 5% CO₂ at 37° C., a DMSO solution of alvocidib was diluted with a medium so that the final concentration of alvocidib would be 0.25 uM and added. After culturing for 1 to 4 days, anti-AR antibodies (Santa Cruz Biotechnology Inc., Cat no. sc-816), anti-pAR^{ser81} antibodies (Merck, Cat no. 04-078), anti-pAR^{ser210+213} antibodies (Abcam, Cat no. ab45089), and anti-beta actin antibodies (Wako, Cat no. 013-24553) were used as primary antibodies to perform immunoblotting by the approach in accordance with Example 1-2.

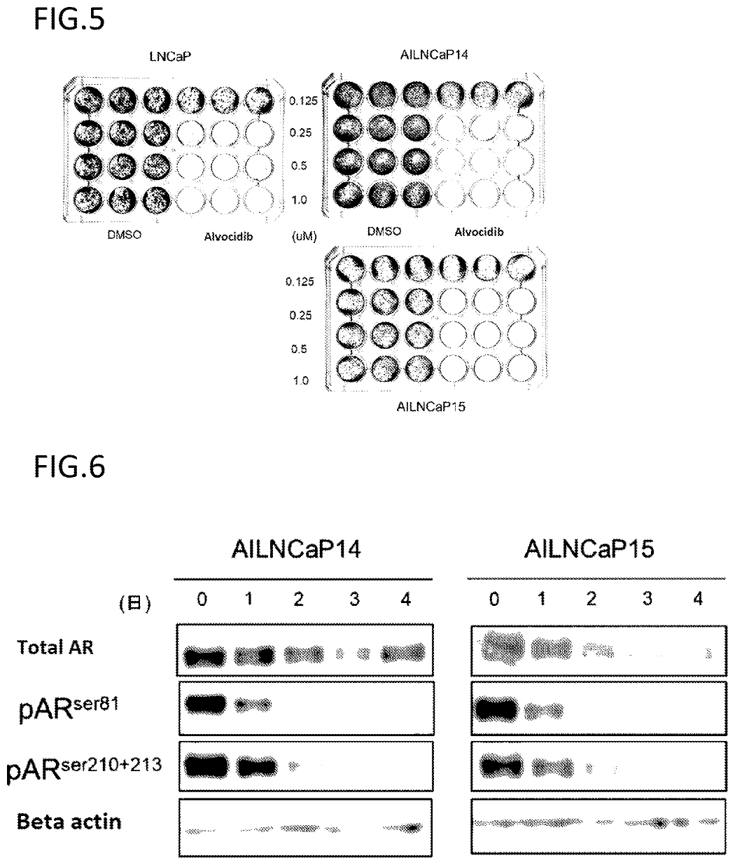

FIG. 6 shows the results of Example 3. It was confirmed that phosphorylation of serine 81, and serine 210 or serine 213 of AR was suppressed over time by allowing 0.25 uM of alvocidib to act on the AILNCaP14 cell line and the AILNCaP15 cell line.

### Example 4

AR Nuclear Translocation Suppression Test with Alvocidib Using Immunofluorescent Staining

1×10⁵ cells of the AILNCaP14 cell line were seeded on a 48-well plate to which 0.5 mL/well of medium was dispensed. After 3 days of culture under the conditions of 5% CO₂ at 37° C., a DMSO solution of alvocidib was diluted with a medium so that the final concentration of alvocidib would be 0.25 μM and added to each well (final volume of 1.0 mL). As a control, alvocidib-free DMSO was similarly diluted and added. After culturing for 4 days, the cells were gently washed once with PBS, and immobilized by allowing 4% paraformaldehyde phosphate buffer to act on the cells for 15 minutes. After washing the cells gently twice with PBS, the cells were incubated for 15 minutes in a 4% PBST solution. The cells were further incubated for 15 minutes in a blocking reagent (DS Pharma Biomedical, Cat no. UK-B80), and the cells were then incubated overnight at 4° C. after adding an anti-AR antibody (Santa Cruz Biotechnology Inc., Cat no. sc-816). After washing the cells gently twice with PBS, the cells were incubated for 45 minutes at room temperature after adding a secondary antibody for immunofluorescent staining (Thermo Fisher Scientific, Cat no A-21206). After washing the cells gently twice with PBS, the cells were incubated for 10 minutes after adding DAPI (Nacalai tesque, Cat no. 11034-56). After washing the cells twice for 10 minutes with 1% PBS, signals were measured with a fluorescence microscope.

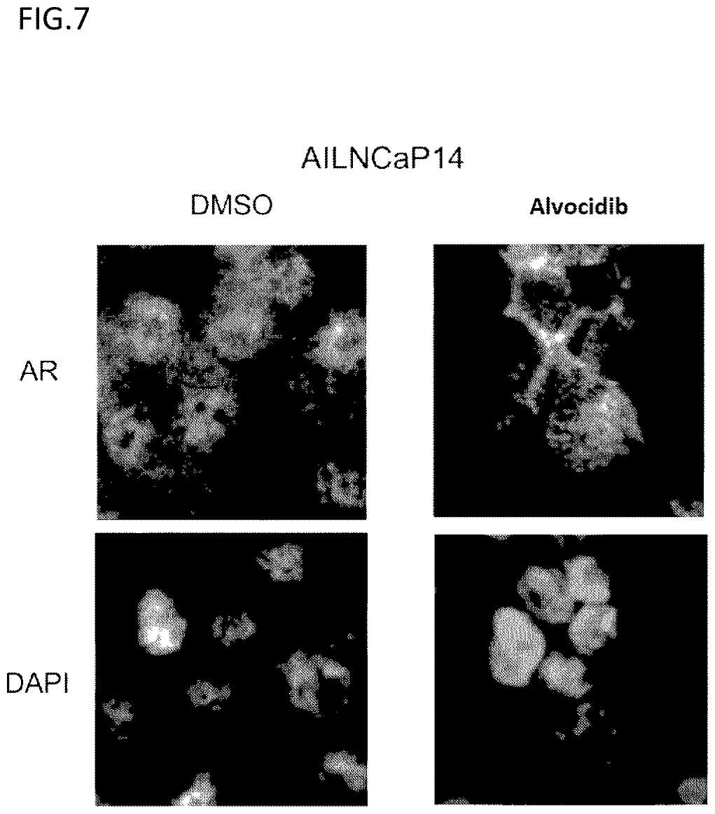

FIG. 7 shows the results of Example 4. It can be understood that the cytoplasm and the nucleus are both stained (cells appear white overall) due to AR staining, so that AR is in both the cytoplasm and the nucleus for the DMSO control (without using alvocidib) in the AILNCaP14 cell line. Meanwhile, it was elucidated that AR staining was in the cytoplasm, but AR staining was reduced in the nucleus (center portion is black and missing) in the AILNCaP14 cell line on which alvocidib was allowed to act. It was found therefrom that AR nuclear translocation was suppressed by treating the AILNCaP14 cell line with alvocidib. Mutant AR was activated constantly and ligand independently in castration resistant prostate cancer resistant to therapy. In view of the above, alvocidib has a significant and different effect of suppressing nuclear translocation of the mutant AR.

Example 5 Tumor proliferation suppression test with alvocidib using a subcutaneous xenograft mouse model

2×10⁶ AILNCaP14 cells were subjected to suspension culture for 4 days using a PrimeSurface® petri dish 90 mm (product code: MS-9090X) in a phenol red free RPMI 1640 medium containing activated carbon-treated 10% fetal bovine serum (csFBS). The cultured cells that were collected, mixed with 350 uL of Matrigel (Corning® Matrigel® Basement Membrane Matrix; catalog number: 356237), and then divided into 3 equal portions were transplanted subcutaneously into three sites on NOD-SCID mice (male, 8 mice, 6 to 8 weeks old) 7 days after castration. After the subcutaneous transplantation, the mice were continuously raised, and an aqueous saline solution of alvocidib hydrochloride (3 mg/kg) was administered intraperitoneally to the mice for a total of three times at an interval of once every two days from the point (day 0) where the volume of tumor formed subcutaneously reached 25 mm³ or greater and 100 mm³ or less at one or two of the tumor transplantation sites.

The tumor volume after three days (day 7) from the third administration was measured, and the ratio of increase in tumor volume was computed in accordance with the following equation.

Ratio of increase in tumor volume=tumor volume on day 7/tumor volume on day 0

As a negative control group, the same test was conducted using saline in place of an agent administered solution, and the ratio of increase in tumor volume was computed.

As a reference, FIG. 9 shows the summary of the test scheme of Example 5.

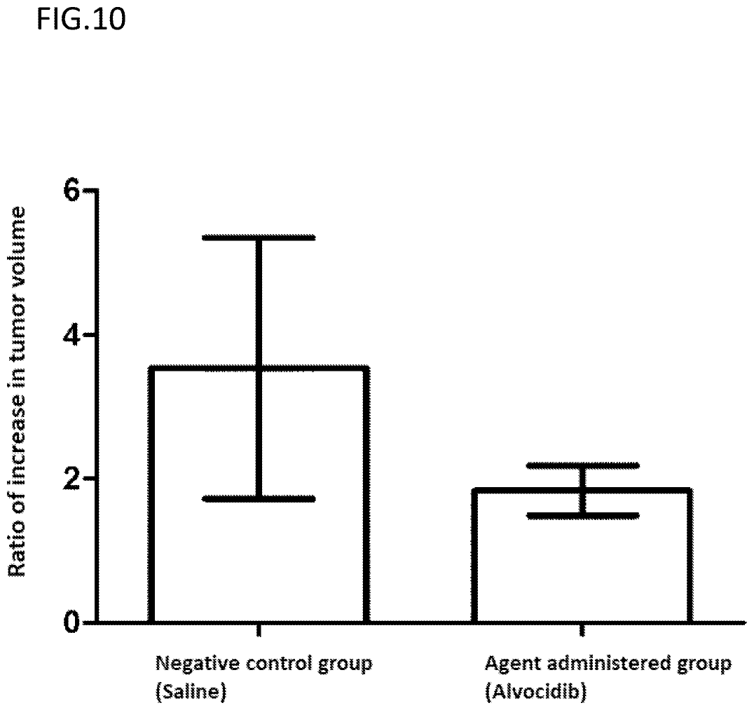

FIG. 10 shows the test results of Example 5. FIG. 10 shows the ratio of increase in tumor volume of an agent administered group as the mean and standard deviation of five mice (six tumors), and the ratio of increase in tumor volume of the negative control group as the mean and standard deviation of three mice (three tumors).

As shown in FIG. 10, the ratio of increase in tumor volume decreased and a higher effect of suppressing tumor proliferation was found with a significant difference (p value=0.0497) in the alvocidib administered group compared to the negative control group. A decrease in the body weight of mice was not observed in either administration group.

The above results support that alvocidib of the invention exhibits a significant and different effect of having a prominent anticancer action, without inducing weight loss, on cancer that exhibits therapeutic resistance to an androgen receptor antagonist and/or an androgen synthesis inhibitor.

### Comparative Example 1

AR Phosphorylation Suppression Test Using Voruciclib

An AR phosphorylation suppression test using voruciclib was conducted in accordance with the experimental procedures of Example 3.

After seeding 2.5×10⁶ cells/well of the AILNCaP14 cell line on a 6-well plate to which 10 mL/well of medium was dispensed and culturing the cells for 6 days under the conditions of 5% CO₂ at 37° C., a DMSO solution of voruciclib (CDK1 or 9 inhibitor) was added so that the final concentrations of voruciclib would be 0.125 uM, 0.25 uM, 0.5 uM, and 1 uM. After culturing for 4 days, anti-AR antibodies (Santa Cruz Biotechnology Inc., Cat no. sc-816), anti-pAR^{ser81} antibodies (Merck, Cat no. 04-078), anti-pAR^{ser210+213} antibodies (Abcam, Cat no. ab45089), and anti-beta actin antibodies (Wako, Cat no. 013-24553) were used as primary antibodies to perform immunoblotting by the approach in accordance with Example 1-2. As a positive control, JQ1 (Cayman Chemical) was used.

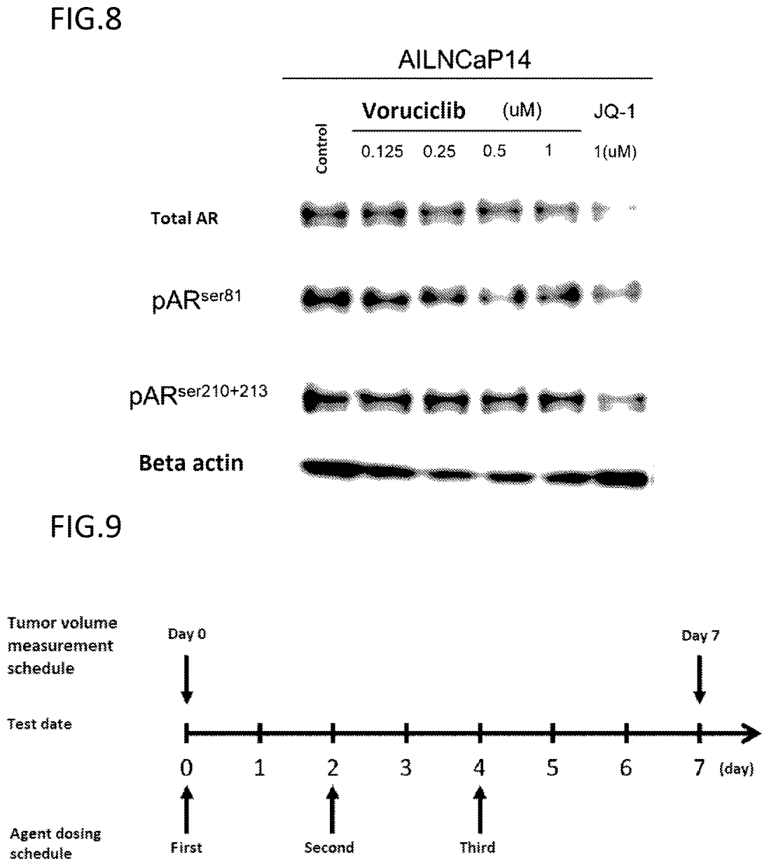

FIG. 8 shows the experimental results of Comparative Example 1. Phosphorylation of Ser81 of AR was slightly suppressed, but phosphorylation of Ser210 or 213 was not suppressed from administration of voruciclib. It is inferred therefrom that the effect of voruciclib in suppressing proliferation of the AILNCaP14 cell line is very low. It was elucidated that alvocidib and voruciclib are both CDK inhibitors, but the effects on the AILNCaP14 cell line are completely different. This means that the efficacy on therapy resistant prostate cancer cannot be predicted even for the same CDK inhibitor.

As disclosed above, the present invention is exemplified by the use of its preferred embodiments. However, it is understood that the scope of the present invention should be interpreted solely based on the Claims. The present application claims priority to Japanese Patent Application No. 2018-244174 (filed on Dec. 27, 2018). The entire content thereof is incorporated herein by reference. It is also understood that any patent, any patent application, and any references cited herein should be incorporated herein by reference in the same manner as the contents are specifically described herein.

### INDUSTRIAL APPLICABILITY

The alvocidib of the present invention or pharmaceutically acceptable salt thereof are extremely useful against “cancer that exhibits resistance to an androgen receptor antagonist or an androgen synthesis inhibitor”.

### Sequence Listing Free Text

SEQ ID NO: 1: AR forward primer
SEQ ID NO: 2: AR reverse primer
SEQ ID NO: 3: AR-V7 forward primer
SEQ ID NO: 4: AR-V7 reverse primer
SEQ ID NO: 5: beta actin forward primer
SEQ ID NO: 6: beta actin reverse primer

1.-15. (canceled)

16. A method for treating cancer in a patient with a serum testosterone concentration reduced to a castration level by castration and/or drug therapy, comprising administering to the patient a composition comprising alvocidib or a pharmaceutically acceptable salt thereof as an active ingredient.

17. A method for treating cancer in a subject with elevated phosphorylation of an androgen receptor, comprising administering to the subject a composition comprising alvocidib or a pharmaceutically acceptable salt thereof as an active ingredient.

18. The method of claim 17, wherein the subject with elevated phosphorylation of an androgen receptor is determined by steps comprising:
(1) quantifying an amount of phosphorylation of an androgen receptor of a cancer cell acquired from the subject;
(2) comparing the amount of phosphorylation quantified in (1) with a control value amount of phosphorylation that has been quantified in a cell collected from a healthy individual; and
(3) determining that phosphorylation is elevated when the amount of phosphorylation quantified in (1) is greater than the control value amount quantified in (2).

19. The method of claim 18, wherein the amounts of phosphorylation in step (1) and the control value amount in step (2) are measured using an anti-androgen receptor antibody.

20. The method of claim 19, wherein the amounts of phosphorylation in step (1) and the control value amount in step (2) are measured using an anti-androgen receptor antibody as a primary antibody, and further using an anti-beta actin antibody.

21. The method of claim 17, wherein the elevated phosphorylation of the androgen receptor comprises phosphorylation of serine 81, and phosphorylation of serine 210 or serine 213 of the androgen receptor.

22. (canceled)

23. The method of claim 17, wherein the cancer is at least one type of cancer selected from acute leukemia, chronic lymphocytic leukemia, chronic myelogenous leukemia, polycythemia vera, malignant lymphoma, plasma cell tumor, multiple myeloma, myelodysplastic syndrome, brain tumor, head and neck cancer, esophageal cancer, thyroid cancer, small cell lung cancer, non-small cell lung cancer, thymoma/thymic carcinoma, breast cancer, gastric cancer, gallbladder/bile duct cancer, liver cancer, hepatocellular carcinoma, pancreatic cancer, colon cancer, rectal cancer, anal cancer, gastrointestinal stromal tumor, choriocarcinoma, endometrial cancer, cervical cancer, ovarian cancer, bladder cancer, prostate cancer, urothelial cancer, renal cancer, renal cell cancer, testicular tumor, testicular germ cell tumor, ovarian germ cell tumor, Wilms tumor, skin cancer, malignant melanoma, neuroblastoma, osteosarcoma, Ewing sarcoma, and soft tissue sarcoma.

24. The method of claim 17, wherein the cancer is prostate cancer, breast cancer, ovarian cancer, or bladder cancer.

25. The method of claim 17, wherein the cancer is prostate cancer.

26. The method of claim 17, wherein the prostate cancer is castration resistant prostate cancer.

27. The method of claim 17, wherein the cancer is characterized by expressing a mutant androgen receptor.

28. The method of claim 27, wherein the mutant androgen receptor is a splicing variant of an androgen receptor.

29. The method of claim 27, wherein the mutant androgen receptor is a splicing variant AR-V7, AR-V12, or AR-V567es.

30. The method of claim 27, wherein the mutant androgen receptor is a splicing variant AR-V7.

31. The method of claim 17, wherein the cancer is cancer that exhibits therapeutic resistance to an androgen receptor antagonist and/or an androgen synthesis inhibitor.

32. The method of claim 31, wherein the androgen receptor antagonist is enzalutamide.

33. The method of claim 31, wherein the androgen synthesis inhibitor is abiraterone.

34. A method of predicting efficacy of alvocidib or a pharmaceutically acceptable salt thereof for treating cancer in a subject, comprising:
(1) quantifying an amount of phosphorylation of an androgen receptor of a cancer cell acquired from the subject;
(2) comparing the amount of phosphorylation quantified in (1) with a control value amount of phosphorylation in a cell collected from a healthy individual; and
(3) determining whether phosphorylation is elevated when the amount of phosphorylation quantified in (1) is greater than the control value amount of (2).

35. (canceled)

36. The method of claim 34, wherein the amounts of phosphorylation in steps (1) and (2) are measured using an anti-androgen receptor antibody.

37. The method of claim 36, wherein the amounts of phosphorylation in steps (1) and (2) are measured using an anti-androgen receptor antibody as a primary antibody, and further using an anti-beta actin antibody.

38. The method of claim 34, wherein the phosphorylation of an androgen receptor is phosphorylation of serine 81, and of serine 210 or serine 213 of the androgen receptor.

39. The method of claim 34, wherein the cancer is at least one type of cancer selected from acute leukemia, chronic lymphocytic leukemia, chronic myelogenous leukemia, polycythemia vera, malignant lymphoma, plasma cell tumor, multiple myeloma, myelodysplastic syndrome, brain tumor, head and neck cancer, esophageal cancer, thyroid cancer, small cell lung cancer, non-small cell lung cancer, thymoma/thymic carcinoma, breast cancer, gastric cancer, gallbladder/bile duct cancer, liver cancer, hepatocellular carcinoma, pancreatic cancer, colon cancer, rectal cancer, anal cancer, gastrointestinal stromal tumor, choriocarcinoma, endometrial cancer, cervical cancer, ovarian cancer, bladder cancer, prostate cancer, urothelial cancer, renal cancer, renal cell cancer, testicular tumor, testicular germ cell tumor, ovarian germ cell tumor, Wilms tumor, skin cancer, malignant melanoma, neuroblastoma, osteosarcoma, Ewing sarcoma, and soft tissue sarcoma.

40. The method of claim 34, wherein the cancer is prostate cancer, breast cancer, ovarian cancer, or bladder cancer.

41. The method of claim 34, wherein the cancer is prostate cancer.

42. The method of claim 41, wherein the prostate cancer is castration resistant prostate cancer.

43. The method of claim 34, wherein the cancer is characterized by expressing a mutant androgen receptor.

44. The method of claim 43, wherein the mutant androgen receptor is a splicing variant of an aldosterone androgen receptor.

45. The method of claim 43, wherein the mutant androgen receptor is a splicing variant AR-V7, AR-V12, or AR-V567es.

46. The method of claim 43, wherein the mutant androgen receptor is a splicing variant AR-V7.

47. The method of claim 34, wherein the cancer exhibits therapeutic resistance to an androgen receptor antagonist and/or an androgen synthesis inhibitor.

48. The method of claim 47, wherein the androgen receptor antagonist is enzalutamide.

49. The method of claim 47, wherein the androgen synthesis inhibitor is abiraterone.

50. A method of treating cancer that exhibits resistance to androgen deprivation therapy, comprising administering alvocidib or a pharmaceutically acceptable salt thereof.

51.-53. (canceled)

54. A kit for use in diagnosing whether a subject is suited to therapy using alvocidib or a pharmaceutically acceptable salt thereof, comprising means for measuring phosphorylation of an androgen receptor of the subject.

55. A cell having androgen non-dependent cell proliferation, wherein the cell is enzalutamide resistant with expression of AR-V7.
